(19) United States
(12) Patent Application Publication
(10) Pub. No.: US 2021/0068483 A1
(43) Pub. Date: Mar. 11, 2021

(54) Passive Nose Warming Apparatus and Safeguard

(71) Applicant: Sean R. Dimsey, Seattle, WA (US)

(72) Inventor: Sean R. Dimsey, Seattle, WA (US)

(21) Appl. No.: 16/563,909

(22) Filed: Sep. 8, 2019

(51) Int. Cl. A41D 31/06; C08L 11/00; A41D 31/02

(52) U.S. Cl. CPC A41D 31/065; A41D 31/02; C08L 11/00

(57) ABSTRACT

A self-adhering, moldable and yet semi-rigid safeguard for the nose that does not require straps or bands, which also simultaneously protects a person's nose from inclement weather.

The apparatus is made of low temperature moldable plastic enclosed between two layers of polychloroprene with a temporary adhesive applied to the back of the attachment so that it may adhere to a person's nose.

The low temperature moldable plastic provides further adherence to a person's nose with its ability to be custom form fitted to a person's exact nose shape because of the safeguard's ability to be molded at warm temperatures.

The low temperature molded plastic then cools to become more rigid offering further protection.

### FIELD OF THE INVENTION

1. Nose Warmer and Nose Protector.

A self adhering, moldable, and semi-rigid protective attachment for the nose that does not require straps or bands and protects against inclement weather.

### BACKGROUND OF INVENTION

Current protective patented apparatuses for the nose provide zero combined moldability and adhesive properties. Current patented apparatuses attach to the nose by means of a strap or band as opposed to this application's use of a low temperature moldable plastic and non-ionic polymer adhesion combination rather than straps, bands or ear supports.

Other patents also lack the proper material to retain warmth as most materials used are highly porous materials that provide little heat retention or adverse weather protection (e.g. fleece or wool).

For example, U.S. Pat. No. 6,070,265 (expired) uses a strap or band to attach a nose protecting apparatus to the person's nose. It is also made from fleece, a highly porous material.

Patent number JP2007098092 uses a chemical reaction for the heating of the nose and large portion of the front facial region; it also envelopes more than just the nose and uses a foldable (not moldable) strap that hugs the side of a person's face.

Further analysis shows that U.S. Pat. No. 5,697,100A (expired) directly attaches to a persons ski goggles using hook type fasteners.

### BRIEF DESCRIPTION OF DRAWINGS

This invention will be described by reference to the following drawings; in which like elements are disclosed by like numerals, and in which:

FIG. 1 is a side view of a person that shows where this invention is placed. It is a protective and passive nose warming apparatus that is moldable and while adhering to a person's nose using a non-ionic polymer and is further held to a person's nose due to the custom fit as provided by the low temperature moldable plastic centered between two layers of polychloroprene.

FIG. 2 is the breakout view of the layers of this inventions protective nose warmer.

FIG. 3 is the breakout view of the layers of the device shaped for a person's nose.

FIG. 4 shows the three layers of material that are to be combined into a single apparatus. Shown in a flat position.

FIG. 5 shows the three layers of the material in a shaped position and combined into a single nose warming and protection apparatus. In a shaped position for a person's nose.

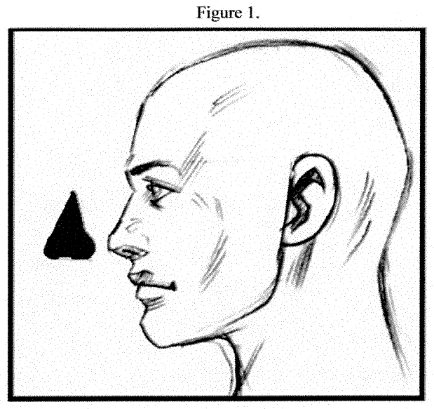

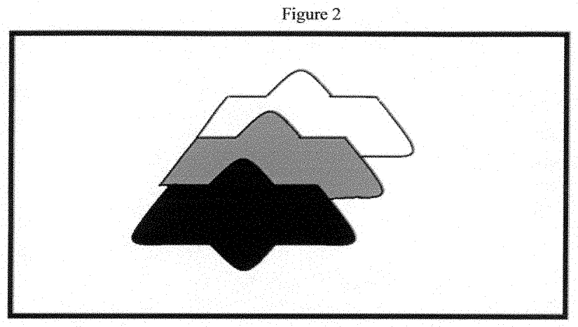

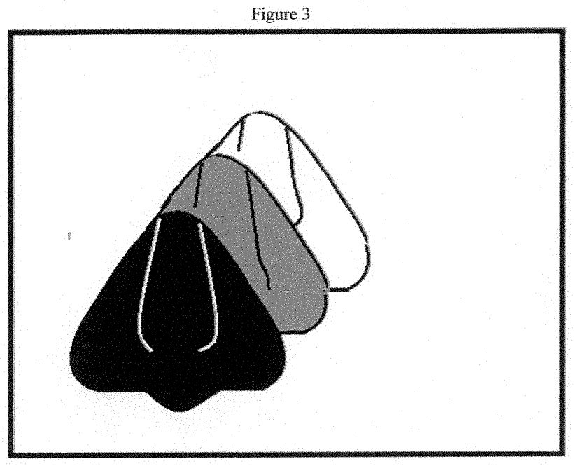

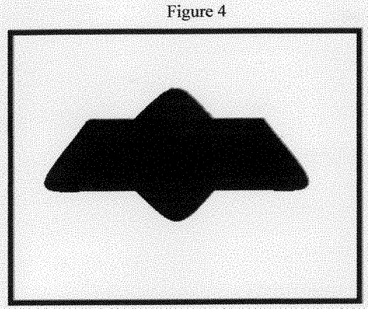

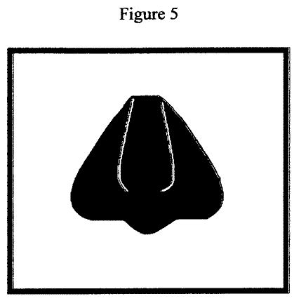

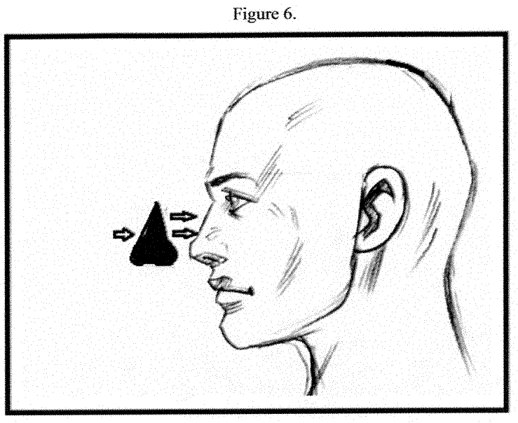

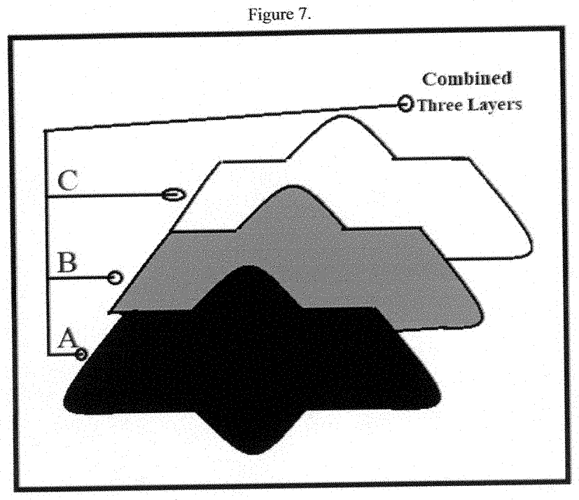

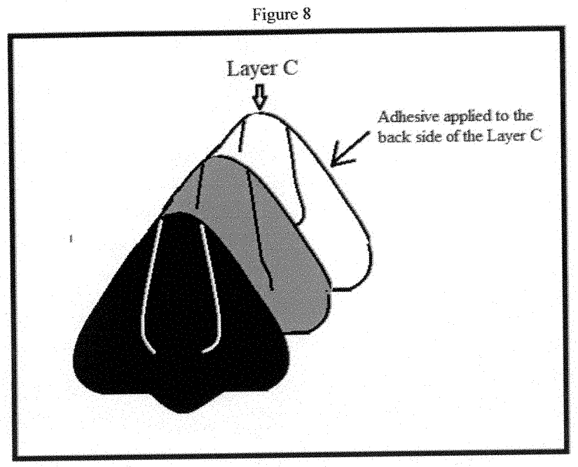

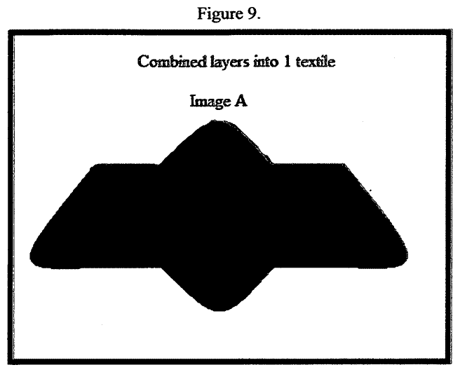

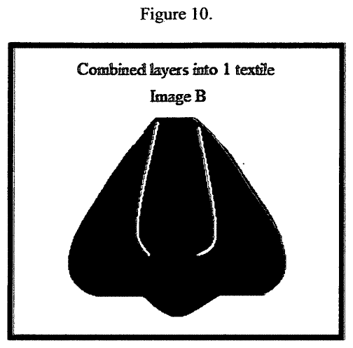

### DESCRIPTION OF THE PREFERRED EMBODIMENTS

FIG. 6 show where the nose warmer adheres to a person's nose. The nose warmer is comprised of two layers of polychloroprene with a low temperature moldable plastic in between the two layers of neoprene.

FIG. 7 identifies the three layers of the apparatus. Layer A is made with polychloroprene which can withstand temperatures from −20 degrees to 180 degrees (Fahrenheit) and is elastic, form fitting and impermeable. Thus, the polychloroprene is well known and is described, e.g., in U.S. Pat. No. 5,773,544A. The disclosure of this U.S. patent is hereby incorporated by reference into this specification. Layer B is made of a low temperature moldable plastic called Polycaprolactone, as described in U.S. Pat. Nos. 5,705,181A and 4,511,687A. Layer C is the same material (polychloroprene) as Layer A.

FIG. 8 shows which layer of the apparatus the nonionic polymer is applied, which is on the back of Layer C. Specifically, the back of Layer C that would be indirect contact with the person's nose. The adhesive adheres the apparatus to a person's nose. The adhesive to be used will be a nonionic polymer as described in U.S. Pat. No. 6,106,818A.

FIG. 9 shows the layers of the apparatus combined or adhered to each other using a polychloroprene adhesive described in U.S. Pat. Nos. 4,603,712A and 3,333,025. Image A is the flat in a flat position.

FIG. 10 shows the layers of the apparatus combined or adhered to each other using a polychloroprene adhesive described in U.S. Pat. Nos. 4,603,712A and 3,333,025. Image B is shown in a shaped position, in the shape of a person's nose.

It is to be understood that the aforementioned description is illustrative only and that changes can be made in the apparatus, in the ingredients and their proportions, and in the sequence of combinations and process steps, as well as in other aspects of the invention discussed herein, without departing from the scope of the invention as defined in the following claims.

1. An adhesive, moldable and semi rigid device for the passive warming and protection of a person's nose that does not require straps or bands. The apparatus is comprised of three layers. The layers are adhered to one another with a polychloroprene adhesive. The invention itself adheres to the person's nose using a temporary nonionic polymer in conjunction with the customized fit gained from the low temperature moldable plastic centered between two layers of polychloroprene.
